(19) United States
(12) Patent Application Publication
(10) Pub. No.: US 2011/0212511 A1
(43) Pub. Date: Sep. 1, 2011

(54) SYSTEM FOR DETECTING BIOMOLECULE WITH HIGH SENSITIVITY USING MICRO-CANTILEVER

(75) Inventors: Kyo Seon Hwang, Icheon-si (KR); Tae Song Kim, Seoul (KR); Sang Kyung Kim, Seoul (KR); Sang Myung Lee, Seoul (KR); Yoon Sik Lee, Anyang-si (KR)

(73) Assignee: KOREA INSTITUTE OF SCIENCE AND TECHNOLOGY, Seoul (KR)

(21) Appl. No.: 13/128,149

(22) PCT Filed: Nov. 11, 2008
(86) PCT No.: PCT/KR2008/006638

§ 371 Date: May 6, 2011

(51) Int. Cl. G01N 33/566

(52) U.S. Cl. 435/287.2; 422/69

(57) ABSTRACT

Provided is a protein detection system using a micro-cantilever and based on immune responses, wherein the micro-cantilever shows significantly improved sensitivity to allow detection of a trace amount of biomolecule. To the micro-cantilever, sandwich immunoassay is applied, and the sandwich immunoassay uses a polyclonal antibody or silica nanoparticles having a monoclonal antibody bound thereto, so that variations in the output signals of the cantilever are amplified and the detection sensitivity is significantly improved. The system enables detection of disease specific antigen at several femtomolar levels, and makes it possible to detect a trace amount of protein related to diseases, particularly to cancers, with ease.

### TECHNICAL FIELD

This disclosure relates to a protein detection system using a micro-cantilever and based on immune responses. More particularly, this disclosure relates to a system for detecting biomolecules using a micro-cantilever, to which sandwich immunoassay is applied, wherein the sandwich immunoassay uses a polyclonal antibody or silica nanoparticles having a monoclonal antibody bound thereto, so that the output signals change of the cantilever are amplified and the detection sensitivity is significantly improved.

### BACKGROUND ART

Micro-cantilevers have been developed in terms of structures and materials along with research and development of microelectromechanical systems (MEMS) and nanoelectromechanical systems (NEMS). In addition, as nanotechnology and biotechnology have received great attention, industrial application of micro-cantilevers has increased dramatically. A micro-cantilever as a biosensor is characterized by high sensitivity, high selectivity and labeling-free detection, and is applied to various analytes, such as DNAs, marker proteins and pathogens including low-molecular-weight biomolecules.

Application of a micro-cantilever sensor is based on the following two main principles: the microbalance principle and the surface stress principle. The former is applied to a dynamic mode, in which variations in resonance frequency represented by a change in the mass and spring constant of a cantilever are measured. The latter is applied to a static mode, in which displacement generated by a change in surface stress derived from a specific response on a micro-cantilever is measured.

When a micro-cantilever functions as a biosensor, there is an opinion about the output signals (variations in resonance frequency or bending degrees) of a micro-cantilever ultimately result from variations in surface stress generated due to the binding of a trace amount of biomolecule to the surface. In other words, it means that when a biomolecule is specifically bound to the surface, a force of interaction between adjacent substances and a structural change caused by the specific binding induce surface stress, which, in turn, causes bending in a micro-cantilever sensor or variations in resonance frequency.

In addition to selectivity and rapidity of a biosensor, sensitivity of a biosensor is one of the most important factors determining the quality of a biosensor. A biosensor generally includes a receptor element for receiving a biomolecule and a transducer element for converting the reception of the biomolecule into electric signals. Recognition of a biomolecule is converted into electric signals by way of an optical or mechanical change. To amplify signals of a biosensor and to improve sensitivity of a biosensor, the transducer element has been improved in electrical and optical aspects, while the receptor element has been improved in chemical and biological aspects.

### DISCLOSURE

### Technical Problem

After conducting many studies, it is found that when sandwich immunoassay using a polyclonal antibody or silica nanoparticles having a polyclonal antibody bound thereto is applied to a micro-cantilever, the cantilever provides amplified variations in output signals, thereby significantly improving the detection sensitivity. Therefore, there is provided a micro-cantilever based system for detecting biomolecules with high sensitivity, which enables detection of biomolecules, such as disease marker proteins, at several femtomolar levels.

### Technical Solution

Disclosed herein is a micro-cantilever sensor based biomolecule detection system, including: a micro-cantilever sensor; a monoclonal antibody layer including a monoclonal antibody against a protein to be detected and formed on the bottom side of the sensor; a protein layer including the protein and formed on the top of the monoclonal antibody layer; and a layer of polyclonal antibody or a layer of polyclonal antibody bound to silica nanoparticles, including a polyclonal antibody against the protein and formed on the top of the protein layer.

The micro-cantilever based biomolecule detection system disclosed herein may further include a self assembled monolayer (SAM) between the micro-cantilever sensor and the monoclonal antibody layer. According to one embodiment of the micro-cantilever based biomolecule detection system disclosed herein, the micro-cantilever sensor may include a lead zirconate titanate (PZT) layer, a thin gold film layer formed on the bottom side of the micro-cantilever sensor, and a self assembled monolayer between the thin gold film layer of the micro-cantilever sensor and the monoclonal antibody layer.

According to another embodiment of the micro-cantilever based biomolecule detection system disclosed herein, a biopolymer for inhibiting non-specific adsorption may be bound onto the monoclonal antibody layer. According to still another embodiment of the micro-cantilever based biomolecule detection system disclosed herein, the polyclonal antibody or the silica nanoparticles may be labeled with a fluorescent material.

### Advantageous Effects

According to the micro-cantilever based biomolecule detection system disclosed herein, it is possible to detect a trace amount of marker protein related to diseases, particularly cancers, with ease at several femtomolar levels.

### DESCRIPTION OF DRAWINGS

The above and other aspects, features and advantages of the disclosed exemplary embodiments will be more apparent from the following detailed description taken in conjunction with the accompanying drawings in which:

FIG. 1 shows schematic views of a micro-cantilever after it performs sandwich immunoassay by using a polyclonal antibody (a) or silica nanoparticles (b) having a polyclonal antibody bound thereto according to one embodiment;

FIG. 2 shows photographs of a micro-cantilever array (a) and an exaggerated micro-cantilever array (b);

FIG. 3 shows a schematic view (a) and transmission electron microscopy (TEM) image of the structure of silica nanoparticles having a size of 140 nm and containing rhodamin B isothiocyanate (RITC) according to one embodiment;

FIG. 4 shows a schematic view (a) of the method for binding RITC to a polyclonal antibody against prostate specific antigen and a schematic view (b) of the method for modifying a polyclonal antibody against prostate specific antigen with RITC-containing silica nanoparticles according to one embodiment;

FIG. 5 shows a graph (a) illustrating variations in resonance frequency of a micro-cantilever at different concentrations of prostate-specific antigen, and fluoroscopic images (b) of the surface of the micro-cantilever at different concentrations of prostate specific antigen, after applying sandwich immunoassay using a polyclonal antibody against prostate specific antigen to the micro-cantilever according to one embodiment;

FIG. 6 shows a graph (a) illustrating variations in resonance frequency of a micro-cantilever at different concentrations of prostate-specific antigen, fluoroscopic images (b) of the surface of the micro-cantilever at different concentrations of prostate specific antigen, and field emission-scanning electron microscopy (FE-SEM) images (c) of the surface of the micro-cantilever at different concentrations of prostate specific antigen, after applying sandwich immunoassay using silica nanoparticles containing a polyclonal antibody against prostate specific antigen bound thereto to the micro-cantilever according to one embodiment; and

FIG. 7 shows flow charts illustrating micro-cantilever based sandwich immunoassay using a polyclonal antibody (a) and the polyclonal antibody (b) modified with silica nanoparticles against prostate specific antigen.

### DETAILED DESCRIPTION OF MAIN ELEMENTS

001, 101: micro-cantilever sensor
002, 102: self-assembled monolayer
003, 103: monoclonal antibody layer and biopolymer layer
004, 104: protein (antigen) to be analyzed
005: fluorescence-labeled polyclonal antibody
105: polyclonal antibody modified with silica nanoparticles

### BEST MODE

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. It will be further understood that the terms “comprises” and/or “comprising” or “concludes” and/or “concluding” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals in the drawings denote like elements. The shape, size and regions, and the like, of the drawing may be exaggerated for clarity.

According to one embodiment, there is provided a method for determining a trace amount of protein with increased sensitivity by amplifying signals of a micro-cantilever biosensor, the method including: (1) providing a resonance type micro-cantilever sensor based on PZT; (2) coating the bottom side of the micro-cantilever sensor with thin gold film and forming a calixcrown self-assembled monolayer on the thin gold film surface; (3) fixing a monoclonal antibody against prostate specific antigen as a bio-receptor on the monolayer, and coating a bovine serum albumin (BSA) layer thereon to inhibit non-specific adsorption; (4) adding prostate specific antigen onto the monoclonal antibody layer so that the antigen is bound thereto; and (5) adding a polyclonal antibody against prostate specific antigen or silica nanoparticles having a polyclonal antibody against prostate specific antigen bound thereto onto the antigen layer so that the polyclonal antibody or the silica nanoparticles having the polyclonal antibody are bound to the antigen layer.

According to one embodiment, the resonance type micro-cantilever based on PZT uses a dynamic mode by which vibration frequency of the cantilever is analyzed. Herein, analyzing the vibration frequency is carried out by measuring variations in phase angles of impedance when a change in surface stress (a change in free energy), generated upon the binding of prostate specific antigen to the monoclonal antibody against prostate specific antigen immobilized to the bottom side of the micro-cantilever, causes a change in natural frequency of the cantilever.

According to another embodiment, a self-assembled monolayer may be used for immobilizing the antibody as a bio-receptor, and the self-assembled monolayer may include a calixcrown compound to which a thiol group is attached. The ether ring structure of calixcrown is capable of capturing positively charged functional groups, such as amine groups. Thus, the calixcrown compound may capture amine groups on the surface of a monoclonal antibody as a bio-receptor so that the antibody is immobilized stably. In addition to calixcrown, 11-mercaptoundecanoic acid or thioctic acid may be used. BSA is used to prevent non-specific adsorption of the surrounding materials mixed with prostate specific antigen, which is the protein to be analyzed, and of the protein to be analyzed itself. Besides BSA, casein may be used.

According to one example embodiment of the method disclosed herein, a micro-cantilever sensor having a monoclonal antibody immobilized thereto as described above is used to capture femtomole-scale prostate specific antigen (PSA) at different concentrations. Generally, prostate specific antigen is a marker protein of prostate cancer and is one of the proteins that have been studied the most intensively as cancer markers. Normal male humans generally have 4 ng/mL or less of prostate specific antigen. Diagnosis of prostate cancer is made at 10 ng/mL or higher. Prostate specific antigen may be expressed even after the surgery of prostate removal, thereby resulting in recurrence of cancer. This may be prevented by early diagnosis through trace analysis. Meanwhile, antigens that may be used herein include alpha-fetoprotein (AFP) as a marker protein of liver cancer, carcinoembryonic antigen (CEA) as a marker protein of colorectal cancer, etc., human epidermal growth factor receptor 2 (HER2) as a marker protein of breast cancer, c-reactive protein (CRP) as a marker protein of cardiovascular diseases, matrix metallopeptidase 9 (MMP-9) as a marker protein of stroke, myoglobin as a marker protein of myocardial infarction, creatine kinase-MB (CR-MB) or troponin-I, or cancer antigen (CA) 19-9, CA 125, RCAS1, TSGF, CA 242, MIC-1, CECAM1 or osteopontin as marker proteins of pancreatic cancer.

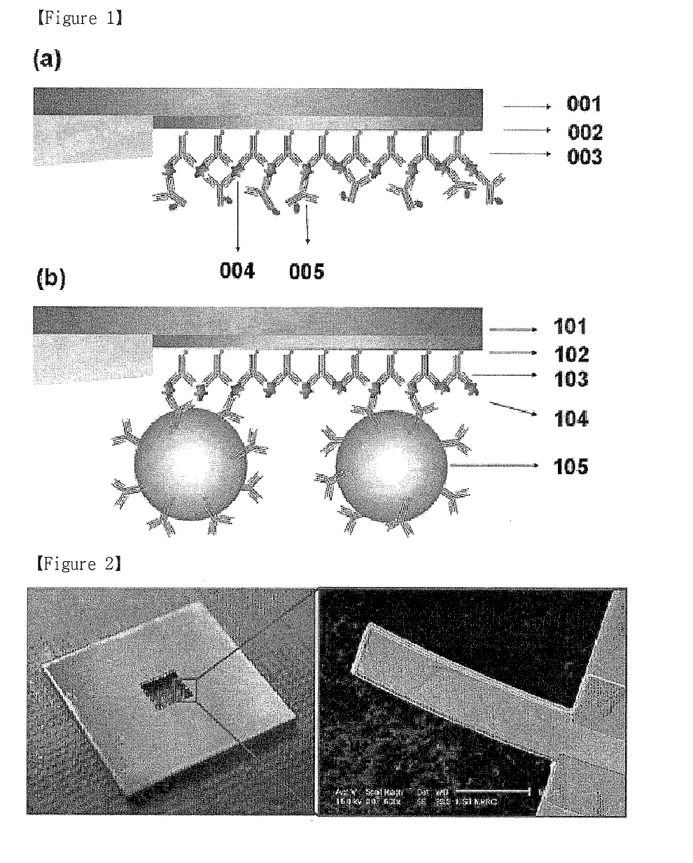

Then, according to one example embodiment of the method disclosed herein, the micro-cantilever in which prostate specific antigen is captured as described above is further subjected to a secondary reaction with a polyclonal antibody against prostate specific antigen, or a polyclonal antibody against prostate specific antigen bound to silica nanoparticles. FIG. 1 is a schematic view of the reacted micro-cantilever, wherein (a) shows the micro-cantilever after carrying out sandwich immunoassay by using the polyclonal antibody, and (b) shows the micro-cantilever after carrying out sandwich immunoassay by using the silica nanoparticle-bound polyclonal antibody against prostate specific antigen. Herein, sandwich immunoassay means a method including generating or amplifying signals through the reaction with a fluorescence dye-, enzyme- or nanoparticle-bound secondary antibody, or a polyclonal antibody, after immune responses.

According to another example embodiment of the method disclosed herein, a rhodamine B isothiocyanate (RITC)-bound polyclonal antibody is used to carry out sandwich immunoassay with a polyclonal antibody against prostate specific antigen. A method for binding RITC to the polyclonal antibody against prostate specific antigen is schematically shown in FIG. 4(a). The RITC-bound polyclonal antibody is used to determine the smooth progress of secondary immune responses using a polyclonal antibody through fluorometry and to perform comparison with amplified signals.

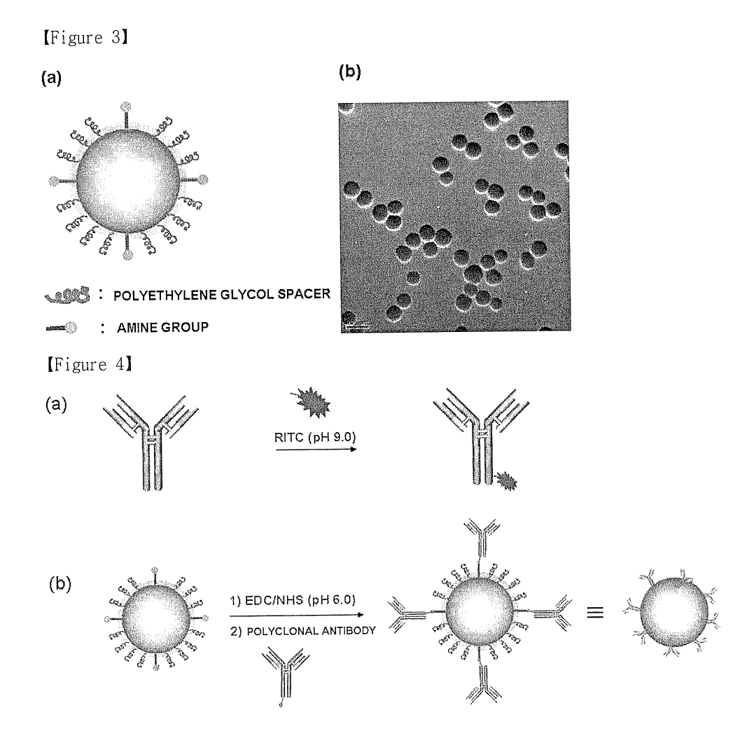

According to still another example embodiment of the method disclosed herein, the Stober method is used to prepare RITC-containing silica nanoparticles so that they may be applied to sandwich immunoassay using a polyclonal antibody against prostate specific antigen bound to silica nanoparticles. To prevent non-specific adsorption, the silica nanoparticles are coated with polyethylene glycol and further coated with amine groups to introduce functional groups. To perform the coating, O-methoxy(polyethyleneoxy)-N-trimethoxysilylpropyl carbamate (M.W. 2175) and 3-aminopropyltriethoxysilane are used in a molar concentration ratio of 5:1. The structure and TEM image of the silica nanoparticles thus prepared are shown in FIG. 3. Then, the amine groups on the surface of the nanoparticles are allowed to react with succinic anhydride to be converted into carboxyl groups, while the polyclonal antibody against prostate specific antigen is immobilized to the silica nanoparticles by using 1-ethyl-3-(3-dimethylaminopropyl)carbodiimide (EDC)/N-hydroxysuccinimide (NHS) through the process as shown in FIG. 4 (b). As in the sandwich immunoassay merely using the polyclonal antibody against prostate specific antigen, in the case of the polyclonal antibody against prostate specific antigen bound to silica nanoparticles, prostate specific antigen is reacted to the micro-cantilever at different concentrations and then secondary immune responses occur. More particularly, the silica nanoparticles may include tetamethoxyorthosilicate, tetraethoxyorthosilicate or sodium silicate, and may have a diameter of 20-2000 nm. Preparation of silica nanoparticles with a diameter less than 20 nm is not allowed. In addition, silica nanoparticles with a diameter greater than 2000 nm is hardly supported by specific binding between protein molecules and frequently causes structural deformation of a cantilever sensor.

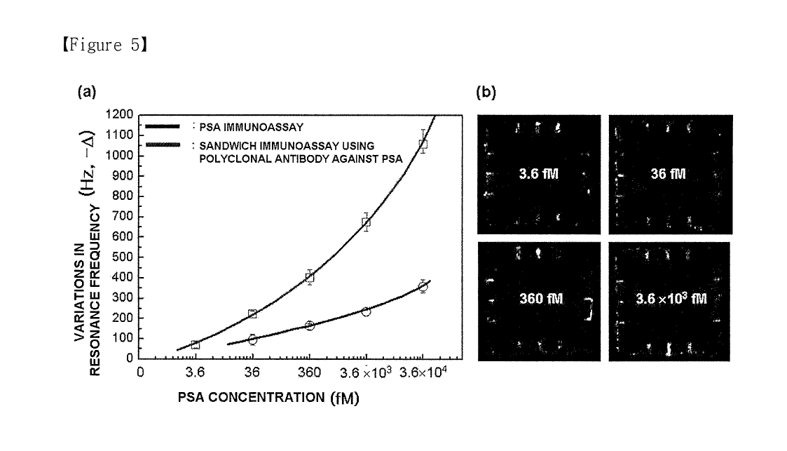

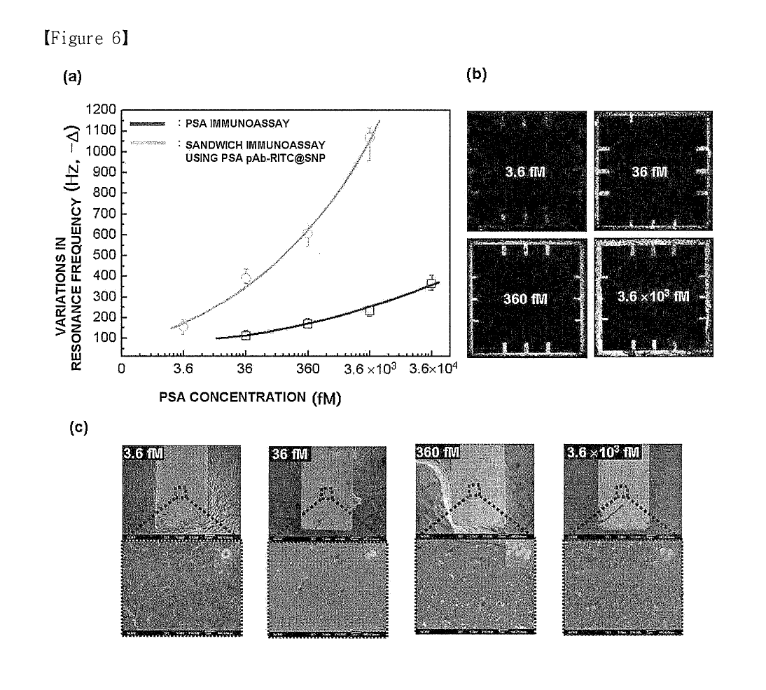

FIG. 5 (detection using the polyclonal antibody) and FIG. 6 (detection using the polyclonal antibody bound to silica nanoparticles) show the results of detection of prostate specific antigen according to one embodiment of the method disclosed herein. Meanwhile, when prostate specific antigen is reacted alone without using sandwich immunoassay with a polyclonal antibody or a polyclonal antibody bound to silica nanoparticles, a quantitatively significant variation is shown in resonance frequency only when the concentration of prostate specific antigen is 360 fM (100 pg/mL) or higher, and variations in resonance frequency at the concentration below the above level are approximately in an error range (see the blue curve in FIG. 5 (a) and the green curve in FIG. 6 (a)). In addition, variations in resonance frequency are not high even at higher concentrations. On the contrary, when amplifying the signals through the sandwich immunoassay using the polyclonal antibody (see the red curve in FIG. 5 (a)), a quantitatively determinable and significant variation is shown in resonance frequency even at a level of 36 fM, and the signals are amplified by 2-3 times at a range of concentration higher than 36 fM. In addition, when amplifying the signals through the sandwich immunoassay using the polyclonal antibody bound to silica nanoparticles (see the pink curve in FIG. 6(a)), a quantitatively determinable and significant variation is shown in resonance frequency even at a level of 3.6 fM, and the signals are amplified by approximately 4 times at a range of concentration higher than 3.6 fM.

Referring to the results of fluorometry, in the case of the sandwich immunoassay using the polyclonal antibody bound to silica nanoparticles, it is shown that secondary immune responses occur proportionally to the concentration of antigen at a range of antigen concentration of 3.6 fM-3.6 pM (see FIG. 6 (b)). It is also determined by scanning electron microscopy (SEM) that the silica nanoparticles are attached to the thin gold film surface on the bottom side of the micro-cantilever proportionally to the concentration of antigen (see FIG. 6 (c)). Meanwhile, when using the polyclonal antibody alone, the results of fluorometry (FIG. 5 (b)) reveal that the intensity of fluorescence is low or does not appear at a range of 3.6 fM-3.6 pM.

### MODE FOR INVENTION

The examples and experiments will now be described. The following examples and experiments are for illustrative purposes only and not intended to limit the scope of this disclosure.

### Example 1

### Sandwich Immunoassay Using Polyclonal Antibody

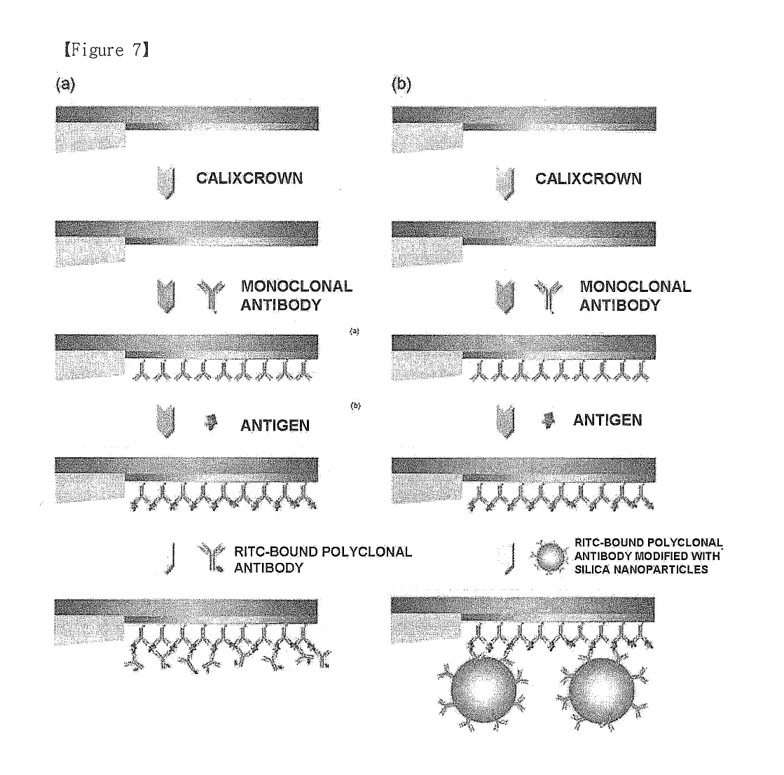

FIG. 7 (a) shows a flow chart of sandwich immunoassay using a polyclonal antibody.

1-1. Immobilization of Monoclonal Antibody Against Prostate Specific Antigen

To form a self-assembled monolayer (SAM) on a thin gold film surface formed on the bottom side of a micro-cantilever, the thin gold film surface is left in 3 mM calixcrown/chloroform solution at room temperature for 2 hours right after the deposition thereof. After the completion of the reaction, the micro-cantilever is washed using chloroform, ethanol and distilled water in turn. To immobilize a monoclonal antibody against prostate specific antigen, the micro-cantilever having the SAM of calixcrown formed thereon is left in 10 μg/mL of aqueous monoclonal antibody against prostate specific antigen/phosphate buffered saline (PBS) solution at room temperature for 1 hour. After the completion of the reaction, the micro-cantilever is washed with PBST (1% Tween 20/PBS) for 15 minutes and then with PBS for 10 minutes. In addition, the micro-cantilever is left in 1% aqueous BSA solution for 1 hour to prevent non-specific adsorption, and washed with PBST (1% Tween 20/PBS) for 15 minutes, with PBS for 10 minutes and then with distilled water for 5 minutes. The resultant micro-cantilever is subjected to measurement of its initial resonance frequency (F₀) by using a micro-cantilever measuring system equipped with a constant-temperature/constant-humidity device.

1-2. Primary Immune Response of Prostate Specific Antigen

Prostate specific antigen is allowed to react with the monoclonal antibody against prostate specific antigen immobilized to the micro-cantilever. The micro-cantilever having the monoclonal antibody immobilized thereto is left in 1 mL of each aqueous solution of prostate specific antigen with concentrations of 3.6 fM (1 pg/mL)-36 pM (10 ng/mL) at room temperature for 1 hour. After the immune reaction, the micro-cantilever is washed with PBST (1% Tween 20/PBS) for 15 minutes, with PBS for 10 minutes and then with distilled water for 5 minutes. Then, the micro-cantilever is subjected to measurement of its resonance frequency (F₁) by using a micro-cantilever measuring system equipped with a constant-temperature/constant-humidity device.

1-3. Sandwich Secondary Immune Responses Using Polyclonal Antibody

To amplify the signals of immune detection responses using a micro-cantilever, a secondary reaction is carried out using a polyclonal antibody. First, the micro-cantilever that has been subjected to the primary immune reaction as described above is left in 1 mL of aqueous solution of a polyclonal antibody against prostate specific antigen modified with 10 μg/mL of rhodamin B isothiocyanate (RITC), at room temperature for 1 hour. After the completion of the reaction, the micro-cantilever is washed with PBST (1% Tween 20/PBS) for 15 minutes, with PBS for 10 minutes and then with distilled water for 5 minutes. Then, the micro-cantilever is subjected to measurement of its resonance frequency (F₂) by using a micro-cantilever measuring system equipped with a constant-temperature/constant-humidity device.

FIG. 5 (a) is a graph showing variations in resonance frequency, i.e. F₁-F₀ in the case of the primary response and F₂-F₀ in the case of the secondary sandwich response, at different concentrations of concentration of antigen. The fluoroscopic image as shown in FIG. 5 (b) may be obtained by using a fluorescent scanner.

### Example 2

### Sandwich Immunoassay Using Polyclonal Antibody Modified with Silica Nanoparticles

FIG. 7 (b) shows a flow chart of sandwich immunoassay using a polyclonal antibody modified with silica nanoparticles.

2-1. Immobilization of Monoclonal Antibody Against Prostate Specific Antigen

To form a self-assembled monolayer (SAM) on a thin gold film surface formed on the bottom side of a micro-cantilever, the thin gold film surface is left in 3 mM calixcrown/chloroform solution at room temperature for 2 hours right after the deposition thereof. After the completion of the reaction, the micro-cantilever is washed using chloroform, ethanol and distilled water in turn. To immobilize a monoclonal antibody against prostate specific antigen, the micro-cantilever having the SAM of calixcrown formed thereon is left in 10 μg/mL of aqueous monoclonal antibody against prostate specific antigen/phosphate buffered saline (PBS) solution at room temperature for 1 hour. After the completion of the reaction, the micro-cantilever is washed with PBST (1% Tween 20/PBS) for 15 minutes and then with PBS for 10 minutes. In addition, the micro-cantilever is left in 1% aqueous BSA solution for 1 hour to prevent non-specific adsorption, and washed with PBST (1% Tween 20/PBS) for 15 minutes, with PBS for 10 minutes and then with distilled water for 5 minutes. The resultant micro-cantilever is subjected to measurement of its initial resonance frequency (F₀) by using a micro-cantilever measuring system equipped with a constant-temperature/constant-humidity device.

2-2. Primary Immune Response of Prostate Specific Antigen

Prostate specific antigen is allowed to react with the monoclonal antibody against prostate specific antigen immobilized to the micro-cantilever. The micro-cantilever having the monoclonal antibody immobilized thereto is left in 1 mL of each aqueous solution of prostate specific antigen with a concentration of 3.6 fM (1 pg/mL)-36 pM (10 ng/mL) at room temperature for 1 hour. After the immune reaction, the micro-cantilever is washed with PBST (1% Tween 20/PBS) for 15 minutes, with PBS for 10 minutes and then with distilled water for 5 minutes. Then, the micro-cantilever is subjected to measurement of its resonance frequency (F₁) by using a micro-cantilever measuring system equipped with a constant-temperature/constant-humidity device.

2-3. Synthesis of Polyclonal Antibody Modified with Silica Nanoparticles

To amplify the signals of immune detection responses using a micro-cantilever, a secondary reaction is carried out using a polyclonal antibody modified with silica nanoparticles. To reduce non-specific binding and to introduce functional groups, 55 mg of fluorescence-containing silica nanoparticles based on tetramethoxyorthosilicate and having a diameter of 140 nm are dispersed into 30 mL of ethanol solution in which 0.01 mmol (220 mg) of O-methoxy(polyethyleneoxy)-N-trimethoxysilylpropylcarbamate (molecular weight: 2175) and 0.02 mmol (4.4 mg) of 3-aminopropyltriethoxysilane are dissolved. Then, 100 μl of aqueous ammonia (25%) is further introduced thereto and agitated at room temperature for 12 hours. After the completion of the reaction, the resultant mixture is washed with ethanol five times for purifying. Thereafter, to convert the amine groups into carboxyl groups, the silica nanoparticles are dispersed into 100 mM succinic anhydride/N,N′-dimethylformamide solution, followed by agitation at room temperature for 2 hours. The silica nanoparticles are washed with dimethylformamide (×3), ethanol (×2) and distilled water (×3) in turn, and then dispersed again into distilled water. Then, to modify the polyclonal antibody against prostate specific antigen with the silica nanoparticles synthesized as described above, the 1-ethyl-3-(3-dimethylaminopropyl)carbodiimide (EDC)/N-hydroxysuccinimide (NHS) coupling method is used. Particularly, 50 mM of EDC and 50 mM of NHS are dissolved in 25 mM of aqueous 2-(N-morpholino)ethanesulfonic acid (MES, pH 6.0) buffer solution, and 1 mg of the silica nanoparticles whose surface groups are converted into carboxyl/polyethylene glycol groups are dispersed. After carrying out reaction with agitation for 1 hour, the reaction mixture is washed with MES buffer once. To the silica nanoparticles thus activated, aqueous solution (1 mL) of 50 μg of the polyclonal antibody against prostate specific antigen is introduced, and reaction is carried out at room temperature for 1 hour, thereby coupling the polyclonal antibody with the silica nanoparticles. After the completion of the reaction, the polyclonal antibody-bound silica nanoparticles are washed with MES solution (×3), PBST solution (×3) and PBS (×2), dispersed again into PBS, and then stored in a refrigerator (4° C.).

2-4. Sandwich Secondary Immune Responses Using Polyclonal Antibody Modified with Silica Nanoparticles

The micro-cantilever that has been subjected to the primary immune reaction as described above is left in 1 mL of aqueous solution of the polyclonal antibody against prostate specific antigen modified with silica nanopartices as described above, at room temperature for 1 hour. After the completion of the reaction, the micro-cantilever is washed with PBST (1% Tween 20/PBS) for 15 minutes, with PBS for 10 minutes and then with distilled water for 5 minutes. Then, the micro-cantilever is subjected to measurement of its resonance frequency (F₂) by using a micro-cantilever measuring system equipped with a constant-temperature/constant-humidity device.

FIG. 6 (a) is a graph showing variations in resonance frequency, i.e. F₁-F₀ in the case of the primary response and F₂-F₀ in the case of the secondary sandwich response, at different concentrations of concentration of antigen. The fluoroscopic image as shown in FIG. 6 (b) may be obtained by using a fluorescent scanner. The surface image of the micro-cantilever surface-coated with silica nanoparticles as shown in FIG. 6 (c) may be obtained by using a field emission scanning electron microscope (FE-SEM) system.

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A micro-cantilever sensor based biomolecule detection system, comprising:
a micro-cantilever sensor;
a monoclonal antibody layer including a monoclonal antibody against the biomolecule to be detected and formed on the bottom side of the sensor;
a biomolecule layer including the biomolecule and formed on the top of the monoclonal antibody layer; and
a layer of polyclonal antibody or a layer of polyclonal antibody bound to silica nanoparticles, including a polyclonal antibody against the biomolecule and formed on the top of the biomolecule layer.

2. The micro-cantilever sensor based biomolecule detection system according to claim 1, which further comprises a self assembled monolayer (SAM) between the micro-cantilever sensor and the monoclonal antibody layer.

3. The micro-cantilever sensor based biomolecule detection system according to claim 1, wherein the micro-cantilever sensor comprises a lead zirconium titanate (PZT) layer, and a thin gold film layer is further formed on the bottom side of the micro-cantilever sensor.

4. The micro-cantilever sensor based biomolecule detection system according to claim 3, which further comprises a self-assembled monolayer between the thin gold film layer of the micro-cantilever sensor and the monoclonal antibody layer.

5. The micro-cantilever sensor based biomolecule detection system according to claim 2, wherein the self-assembled monolayer comprises at least one selected from the group consisting of calixcrown, 11-mercaptoundecanoic acid and thioctic acid.

6. The micro-cantilever sensor based biomolecule detection system according to claim 1, wherein a biopolymer for inhibiting non-specific binding is bound onto the monoclonal antibody layer.

7. The micro-cantilever sensor based biomolecule detection system according to claim 6, wherein the biopolymer for inhibiting non-specific binding is at least one selected from the group consisting of bovine serum albumin (BSA) and casein.

8. The micro-cantilever sensor based biomolecule detection system according to claim 1, wherein the polyclonal antibody or silica nanoparticle is labeled with a fluorescent material.

9. The micro-cantilever sensor based biomolecule detection system according to claim 1, wherein the biomolecule is a disease marker protein, and the disease marker protein is at least one selected from the group consisting of alpha-fetoprotein (AFP), carcinoembryonic antigen (CEA), human epidermal growth factor receptor 2 (HER2), prostate specific antigen (PSA), c-reactive protein (CRP), matrix metallopeptidase 9 (MMP-9), myoglobin, creatine kinase-MB (CK-MB), troponin-I, cancer antigen (CA) 19-9, CA 125, RCAS1, TSGF, CA 242, MIC-1, CECAM1 and osteopontin.

10. The micro-cantilever sensor based biomolecule detection system according to claim 1, wherein the silica nanoparticle comprises tetramethoxyorthosilicate, tetraethoxyorthosilicate or sodium silicate, and has a diameter of 20-2000 nm.

11. The micro-cantilever sensor based biomolecule detection system according to claim 4, wherein the self-assembled monolayer comprises at least one selected from the group consisting of calixcrown, 11-mercaptoundecanoic acid and thioctic acid.
